(19) United States
(12) Patent Application Publication
(10) Pub. No.: US 2025/0121143 A1
(43) Pub. Date: Apr. 17, 2025

(54) Universal Atomizer

(71) Applicant: Innovate (Wuxi) Medical Technology Co., LTD, Wuxi City (CN)

(72) Inventors: Qi DING, Wuxi City (CN); Xueqing ZU, Wuxi City (CN); Chunxiao HU, Wuxi City (CN); Bo WU, Wuxi City (CN); Xiaoshan LI, Wuxi City (CN); Hong GAO, Wuxi City (CN)

(21) Appl. No.: 18/912,610

(22) Filed: Dec. 2, 2024

(30) Foreign Application Priority Data
Oct. 13, 2023 (CN) 202311324938.4
Jun. 20, 2024 (CN) 202421409839.6

(51) Int. Cl. A61M 11/00; A61M 15/00

(52) U.S. Cl. CPC A61M 11/00; A61M 15/0001; A61M 15/0063

(57) ABSTRACT

An atomizer includes an atomization assembly and a medicine storage assembly idetachably coupled to the atomization assembly, wherein the medicine storage assembly includes a storage housing, and a medicine liquid transporting cotton disposed in the storage housing, wherein the atomization assembly includes a fixing housing and an atomizing element disposed in the fixing housing to align with the medicine liquid transporting cotton in a manner that the medicine liquid transporting element is arranged to deliver medicine liquid to the atomizing element.

### CROSS REFERENCE OF RELATED APPLICATION

This application is a non-provisional application that claims priority under 35 U.S.C. § 119 to China application number CN202311324938.4, filing date Oct. 13, 2023, and China application number CN202421409839.6, filing date Jun. 20, 2024, wherein the entire content of which is expressly incorporated herein by reference.

### BACKGROUND OF THE PRESENT INVENTION

### Field of Invention

The present invention relates to atomizer, and more particularly to a core-replaceable atomizer.

### Description of Related Arts

A medical nebulizer atomizes the liquid medicine and then enters the patient's body through the respiratory tract, thus achieving the purpose of atomization treatment.

The liquid medicine in the current nebulizer relies on gravity to contact the atomizer sheet in the nebulizer, which means that there are requirements for the placement of the nebulizer, such as position and angle. That is, the nebulizer needs to be placed in a standing position. Once it is inverted and deviated, the nebulizer will not work properly.

At the same time, in actual use, some patients need more than one medicine for nebulization treatment. For example, upper respiratory tract infection requires medicines such as budesonide or ambroxol. If there is subsequent inflammation, additional dextromethorphan or pentoxyverine cough suppressant may be needed for treatment. If there is sputum, cholinergics may also be needed.

Different medicines need to be used separately to achieve different treatment purposes (some hospitals will require that the nebulizer be replaced entirely). In this process, apart from the cost and cumbersome operation, the removal of the nebulizer itself will cause the breathing circuit to be interrupted for a long time, which may even affect the patient's life safety, especially in the ICU or hyperbaric oxygen chamber. There is a potential risk of cross-infection of bacteria (especially super bacteria). Once infected, the consequences are extremely serious.

In addition, the conventional nebulizers need to be reused many times due to cost and other reasons, but cleaning and disinfection are cumbersome. If the cleaning is not thorough, the mixing of different medicines will also affect the safe use of the medicines themselves.

If there is no filter element in the medicine storage device, impurities generated during the medicine preparation process, such as glass ampoules or vial plugs, can easily cause the mesh pores of the nebulizer to become clogged, affecting the normal atomization of the nebulizer and even shortening its service life.

### SUMMARY OF THE PRESENT INVENTION

The invention is advantageous in that it provides a core-changeable atomizer comprising a medicine module and a control module, wherein the medicine module and the control module are detachably connected and fixed, and the control module controls the atomization release of the medicine in the medicine module.

The control module comprises a control unit and a fixing housing which is provided with a medicine module accommodating chamber and a mist outlet.

The medicine module comprises a medicine containing cavity, a medicine injection structure located at one end of the medicine module, and a medicine liquid transporting cotton accommodated in the medicine containing cavity, wherein a liquid outlet is arranged near the mist outlet of the medicine liquid transporting cotton.

The medicine module is matched with the medicine module accommodating chamber; an atomizing element is provided between the liquid outlet and the mist outlet; the atomizing element is electrically connected to and controlled by the control unit; the medicine liquid in the medicine containing cavity fills the medicine liquid transporting cotton and contacts the atomizing element.

Preferably, the atomizing element is fixed in the fixing housing and is located below the medicine module accommodating chamber, and sealing rings are provided on both the upper and lower sides of the atomizing element.

Preferably, the atomizing element is provided at one end of the medicine module, and the atomizing element is detachably electrically connected to the control unit.

Preferably, the atomizing element and the control unit may be electrically connected in a contact manner or in an insertion manner.

Preferably, the distance between the medicine liquid transporting cotton and the atomizing element is less than 0.3 mm.

Preferably, one end of the medicine module away from the medicine injection structure is coupled to the fixing housing via a threads.

Alternatively, the end of the medicine module away from the medicine injection structure is connected to the fixing housing in a limiting and sealing manner via a sealing ring.

Preferably, a sealing device is also provided at the bottom of the medicine module.

Preferably, the fixing housing is further provided with an air exhaust structure having an exhaust hole located on the outer wall of the fixing housing, an air retention area located at the exhaust hole, and a water-blocking and breathable membrane located between the exhaust hole and the air retention area.

Preferably, a sealing plug is further provided at the top of the medicine module, and a transparent observation window is further provided on the outer side of the control module.

The above core-changeable atomizer of the present invention has the following advantages.

It gets rid of the situation that the normal operation of conventional atomizers is affected by gravity factors, and there is no need to deliberately place the atomizer in a position that meets the requirements of the gravity line.

By replacing the medicine module, the medicine that needs to be atomized can be replaced conveniently and quickly, and the control module can be retained and reused, saving patients' expenses.

Multiple doses of nebulized medicines can be prepared in advance, and suitable medicines can be directly taken for patients to use according to the needs of different patients, thereby improving the work efficiency of medical staff and reducing the time patients wait for treatment.

The medicine module is disposable after use and does not require cleaning or disinfection, thus avoiding cross infection among patients in the hospital.

The present invention further provides a universal atomizer comprising a liquid medicine storage assembly and an atomization generating assembly.

The liquid medicine storage assembly has a built-in filter element for guiding and filtering the liquid medicine, and one end of the atomization generating assembly is connected to the liquid medicine storage assembly, and the other end is connected to the patient's inhalation end;

The atomization generating assembly comprises a base for supporting the atomization element and a connecting tube connected to the patient's inhalation end. One end of the base is connected to the liquid medicine storage assembly, and the other end is provided with an atomization area for placing the atomizing element. One end of the connecting tube is fixed by connecting to the base to limit the atomizing element, and the other end is an interface connected to the patient's inhalation end.

Preferably, the liquid medicine storage assembly comprises a shell connected and fixed to the base, and a drug injection hole is arranged at the rear of the shell, and a sealing cover is arranged at the drug injection hole.

Preferably, the liquid medicine storage assembly and the atomization generating assembly are detachably connected, and a plurality of groups of detachable fixing structures are arranged at the connection between the shell of the liquid medicine storage assembly and the base of the atomization generating assembly, each group of the fixing structures comprises an “L”-shaped adapter block located on the shell and an embedding area located on the base, and the positions of the adapter blocks and the embedding areas in each group of fixed structures correspond to each other.

Preferably, sealing rings are provided on the upper and lower sides of the atomizing element.

Preferably, a plurality of embedded legs are provided at equal angles on one side of the connecting pipe close to the base, and are fixed in position after penetrating the base.

Preferably, the atomizing element is connected to an external power source through a connection port on the side wall of the base.

Preferably, the atomizing element adopts an atomizing sheet.

The universal atomizer of the present invention has the following advantages.

The filter element adsorbs the liquid medicine, ensuring that the liquid medicine is always in contact with the atomizing element, promoting continuous atomization of the medicine and getting rid of the reliance on gravity.

Impurities generated during the preparation of the drug solution, such as debris such as glass ampoules or vial plugs, are filtered through the filter element to prevent the microporous mesh screen of the atomizer from being clogged, affecting the normal atomization work of the atomizer and even shortening its service life.

The nebulized medicine can be replaced simply by replacing the liquid medicine storage assembly, eliminating the cleaning and disinfection process of the previous plan. The operation is simple, and the medicine can be prepared in advance, which improves the work efficiency of medical staff and reduces cross-infection in the hospital.

The present invention further provides an atomizer, comprising:
a medicine storage assembly; and
an atomization assembly, wherein the medicine storage assembly is detachably coupled to the atomization assembly.

According to an embodiment, the medicine storage assembly comprises an outer housing having a storage cavity, and a medicine liquid transporting element disposed in the storage cavity of the outer housing, wherein the atomization assembly comprises an atomizing element to align with the medicine liquid transporting element in a manner that the medicine liquid transporting element is arranged to deliver medicine liquid to the atomizing element.

According to an embodiment, the atomization assembly further comprises a base and a connecting tube detchably coupled to the base, wherein the atomizing element is mounted on the base.

According to an embodiment, the connecting tube is detachably coupled with the outer housing for supporting the outer housing.

According to an embodiment, the connecting tube is coupled at a position between the outer housing and the base.

According to an embodiment, the medicine liquid transporting element is a cotton.

According to an embodiment, the medicine liquid transporting element is a cotton which comprises a body portion and an end portion extended from the body portion, wherein the outer housing has an opening, the end portion of the medicine liquid transporting element is extended through the opening which is communicated to the storage cavity of the outer housing for feeding the medicine liquid to the atomizing element.

According to an embodiment, a diameter of the end portion of the medicine liquid transporting element is smaller than or equal to a diameter of the body portion of the medicine liquid transporting element.

According to an embodiment, a distance between the end portion of the medicine liquid transporting element and the atomizing element is less than 0.3 mm.

According to an embodiment, the medicine storage assembly further comprises a medicine injection structure which comprises an injection lid mounted to a top of the outer housing for refilling, a connecting element connected to the injection lid and a sealing pin extended from the connecting element for being inserted into the injection lid.

According to an embodiment, the medicine storage assembly comprises a storage housing having a storage cavity, and a medicine liquid transporting element disposed in the storage cavity of the storage housing, wherein the atomization assembly comprises a fixing housing and an atomizing element disposed in the fixing housing to align with the medicine liquid transporting element in a manner that the medicine liquid transporting element is arranged to deliver medicine liquid to the atomizing element, wherein the storage housing is detachably coupled within the fixing housing.

According to an embodiment, the fixing housing comprises a base and a housing body coupled to the base, wherein the atomizing element is mounted on the base.

According to an embodiment, the storage housing is detachably disposed in the housing body.

According to an embodiment, a side chamber is formed between the storage housing and the housing body, wherein the atomization assembly comprises a control unit which is disposed in the side chamber.

According to an embodiment, the atomizing element is electrically and detachably connected to the control unit.

According to an embodiment, the medicine liquid transporting element is a cotton.

According to an embodiment, the medicine liquid transporting element is a cotton which comprises a body portion and an end portion extended from the body portion, wherein the housing body has an opening, the end portion of the medicine liquid transporting element is extended through the opening which is communicated to the storage cavity of the storage housing for feeding the medicine liquid to the atomizing element.

According to an embodiment, a diameter of the end portion of the medicine liquid transporting element is smaller than or equal to a diameter of the body portion of the medicine liquid transporting element, wherein a distance between the end portion of the medicine liquid transporting element and the atomizing element is less than 0.3 mm.

According to an embodiment, the storage housing is further provided with an air exhaust structure having an exhaust hole located at a top of the storage housing and an air retention area located adjacent to the exhaust hole, wherein the air exhaust structure further comprises a water-blocking and breathable membrane located between the exhaust hole and the air retention area.

According to an embodiment, the medicine storage assembly further comprises a medicine injection structure which comprises an injection lid mounted to a top of the outer housing for refilling, a connecting element connected to the injection lid and a sealing pin extended from said connecting element for being inserted into said injection lid.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a core-replaceable atomizer according to a first preferred embodiment of the present invention.

FIG. 2 is a sectional view along line A-A of FIG. 1.

FIG. 3 is a sectional view along line B-B of FIG. 1.

FIG. 4 is a partial enlarged view of area C of FIG. 1.

FIG. 5 is a schematic view an alternative medicine module of the core-replaceable atomizer according to the first preferred embodiment of the present invention.

FIG. 6 is a schematic view an alternative control module 2 of the core-replaceable atomizer according to the first preferred embodiment of the present invention.

FIG. 7 is a schematic view illustrating the alternative core-replaceable atomizer being in a state of after assembly.

FIG. 8 is a perspective view of a core-replaceable atomizer according to a second preferred embodiment of the present invention.

FIG. 9 is a sectional view of the core-replaceable atomizer according to the second preferred embodiment of the present invention.

FIG. 10 is a schematic view illustrating an atomization generating assembly of the core-replaceable atomizer according to the second preferred embodiment of the present invention.

FIG. 11 is an exploded view of illustrating the atomization generating assembly of the core-replaceable atomizer according to the second preferred embodiment of the present invention.

FIG. 12 is a perspective view of an atomizer according to a third preferred embodiment of the present invention.

FIG. 13 is a sectional view of the atomizer according to the third preferred embodiment of the present invention.

FIG. 14 is an exploded view of the atomizer according to the third preferred embodiment of the present invention.

FIG. 15 is a perspective view of an atomizer according to a fourth preferred embodiment of the present invention.

FIG. 16 is an exploded view of the atomizer according to the fourth preferred embodiment of the present invention

FIG. 17 is a sectional view of the atomizer according to the fourth preferred embodiment of the present invention.

FIG. 18 is another sectional view of the atomizer according to the fourth preferred embodiment of the present invention.

FIG. 19 is an enlarged perspective view of a medicine storage assembly of the atomizer according to the fourth preferred embodiment of the present invention.

FIG. 20 is an enlarged sectional view of the medicine storage assembly of the atomizer according to the fourth preferred embodiment of the present invention.

FIG. 21 is an enlarged sectional view of an air exhaust structure of the medicine storage assembly of the atomizer according to the fourth preferred embodiment of the present invention.

FIG. 22 is a sectional view of the atomizer according to an alternative mode of the fourth preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art.

The general principles defined in the following description would be applied to other embodiments, alternatives, modifications, equivalents, and applications without departing from the spirit and scope of the present invention.

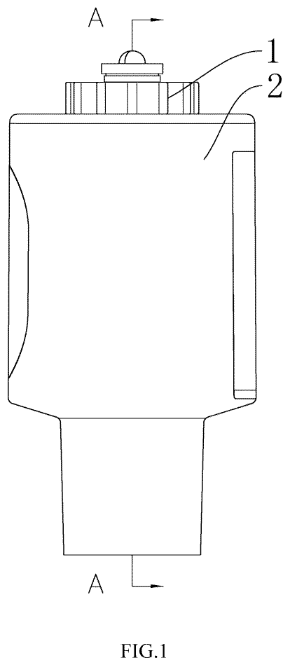

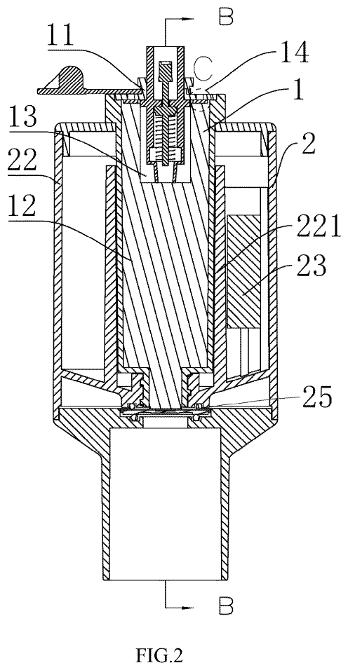

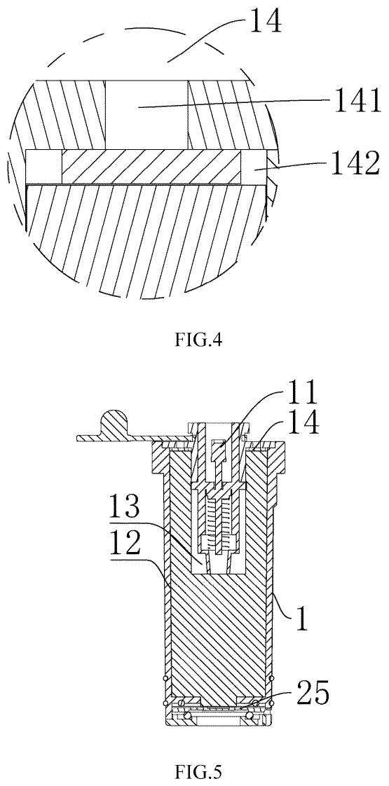

As shown in FIGS. 1 to 4, the core-replaceable atomizer according to a first preferred embodiment of present invention comprises a medicine module 1 and a control module 2, wherein the medicine module 1 and the control module 2 are detachably connected and fixed, and the control module 2 controls the atomization release of the medicine in the medicine module 1.

This method is simple and quick to operate, and can realize the process of pre-dispensing-selection-use, which is extremely efficient, especially for some commonly used nebulized medicines, such as budesonide, ipratropium bromide, acetylcysteine, a certain amount can be pre-dispensed to reduce the waiting time for treatment when patients are densely populated.

The control module 2 comprises a control unit 23 and a fixing housing 22 which is formed with an accommodating chamber 221 and a mist outlet.

The control unit 23 comprises an electrically connected control panel and a power supply, wherein the power supply is fixed in a side cavity connected to the fixing housing 22, and the control panel is located on a side wall connected to the fixing housing 22. The control panel can be used to control the opening and closing of the nebulizer, and the time for the patient to receive nebulization treatment can also be controlled by setting a time length, thereby controlling the time for the patient to absorb the medicine, so as to meet the purpose of different treatment doses for different patients.

In addition, the structure of the medicine module 1 is as follows.

The medicine module 1 has a medicine containing cavity 13, a medicine injection structure 11 located at one end of the medicine module 1, and a medicine liquid transporting cotton 12 accommodated in the medicine containing cavity 13, wherein a mist outlet is arranged near the liquid outlet of the medicine liquid transporting cotton 12.

The medicine injection structure 11 can be a conventional medical injection valve for injecting liquid medicine and preventing it from flowing out, which will not be described in detail here. The medicine liquid transporting cotton 12 is made of medical cotton (generally polyester fiber), which has a porous physical structure. When such cotton encounters liquid, due to its own porous structure, the liquid medicine will flow and be adsorbed to a balanced state (capillary phenomenon) inside it, so that the medicine liquid transporting cotton 12 as a whole always maintains a “filled” state of liquid medicine.

The medicine module 1 is matched with the accommodating chamber 221, facilitating installation and replacement of the medicine module 1; an atomizing element 25 is provided between the liquid outlet and the mist outlet; the atomizing element 25 is electrically connected to and controlled by the control unit 23; the medicine liquid in the medicine containing cavity 13 fills the medicine liquid transporting cotton 12 and contacts the atomizing element 25; the contact method is based on the physical properties of the medicine liquid transporting cotton 12 itself.

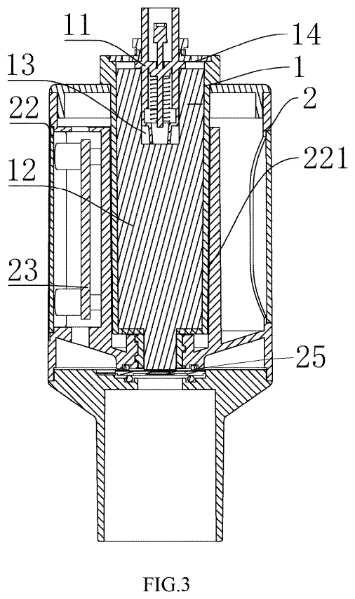

Referring to FIG. 3, a method of electrically connecting the atomizing element 25 and the control unit 23 is as follows.

The atomizing element 25 is arranged in the fixing housing 22 and is located below the medicine module accommodating chamber 221. Sealing rings are provided on both the upper and lower sides of the atomizing element 25.

In this embodiment, the medicine liquid transporting cotton 12 inside the medicine containing cavity 13 directly contacts the atomizing element 25.

The atomizing element 25 adopts an atomizing sheet with pores which works by vibration. The sealing ring provides a working space for the atomizing sheet and also seals the atomized liquid. Different models of atomizing sheets can determine the atomized particle size, atomization efficiency, etc.

Taking the use of budesonide as an example, budesonide suspension 1 mg/2 ml needs to be mixed with 1-2 ml of normal saline and then nebulized.

Under normal circumstances, the dosage of budesonide nebulization is 1-2 mg for adults, twice a day; 0.5-1 mg for children, twice a day. In actual use, the actual inhaled dose of patients during the 15-20 minutes of nebulization is 40-60% of the labeled amount.

In this implementation, since the medicine liquid transporting cotton 12 itself has a certain adsorption to the medicine, the actual inhaled dose is 20 to 50% of the labeled amount, that is, additional equipment is required that exceeds the conventional amount by 10% to 20% (Generally, 10% is enough.) After multiple tests, after atomization, the concentration of the atomized liquid will continue to reach the standard within 30 seconds, and the atomization efficiency of the medicine will be significantly low after 25 minutes.

According to the atomization index, a single atomization time is within 15-20 minutes, which will not affect normal use.

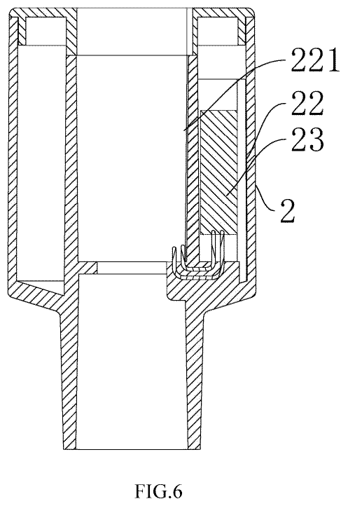

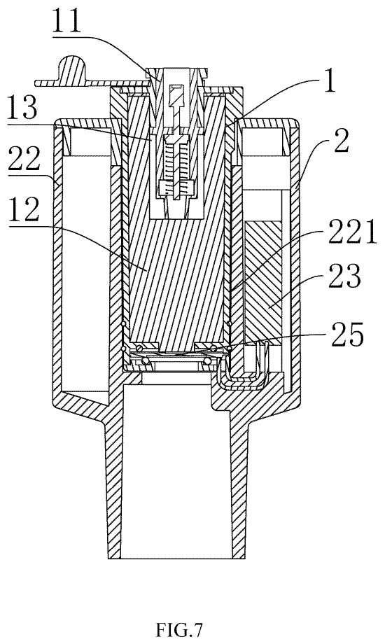

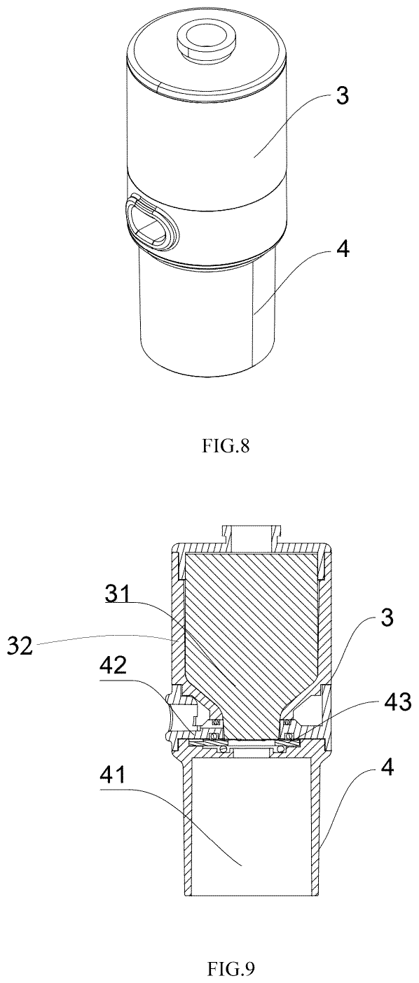

Referring to FIGS. 5, 6 and 7, another way of electrical connection between the atomization element 25 and the control unit 23 is described as follows.

The atomizing element 25 is provided at the bottom of the medicine module 1, and the atomizing element 25 and the control unit 23 are detachably electrically connected.

The atomizing element 25 and the control unit 23 may adopt contact electrical communication or plug-in electrical communication.

In this embodiment, the atomizing element 25 is arranged at one end of the medicine module 1, and the atomizing sheet is still used. The medicine liquid transporting cotton 12 is directly attached to the atomizing sheet, and the medicine liquid in the medicine liquid transporting cotton 12 is continuously atomized through the atomizing sheet.

In the above two connection manners, the medicine module 1 for configuring medicines in the first connection manner is disposable and discarded after use, while the control module 2 needs to be prepared for different patients and can be reused.

The medicine module 1 of the second connection manner can be reused by a dedicated person, and the control module 2 can also be reused.

At the same time, in order to ensure that the liquid medicine in the medicine liquid transporting cotton 12 can be smoothly atomized by the atomizing element 25, the distance between the medicine liquid transporting cotton 12 and the atomizing element 25 is less than 0.3 mm, so as to ensure that the atomizing element 25 works normally, that is, when the atomizing sheet vibrates, the liquid medicine in the medicine liquid transporting cotton 12 and the medicine containing cavity 13 can pass through the pores of the atomizing sheet smoothly and be atomized into small water droplets, thereby achieving the purpose of atomization treatment.

As a connection manner between the medicine module 1 and the fixing housing 22, the portion of the medicine module 1 away from the medicine injection structure 11 is coupled to the fixing housing 22 via threads.

Alternatively, the portion of the medicine module 1 away from the medicine injection structure 11 is connected to the fixing housing 22 in a limiting sealing manner via a sealing ring.

The former connecting manner is conducive to the regulation of the distance, that is, the control of the distance between the medicine liquid transporting cotton 12 and the atomizing element 25, while the latter is convenient for insertion and removal, thus improving the work efficiency of medical staff and having a better sealing effect.

In order to meet the requirements of pre-configuration, a sealing device is also provided at the bottom of the medicine module 1, which serves to isolate the inside and outside of the medicine after the medicine is injected, thereby preventing the medicine from being contaminated. In this embodiment, the sealing device is in the form of a sealing cover.

The fixing housing 22 is further provided with an air exhaust structure 14 having an exhaust hole 141 located at the outer wall of the fixing housing 22, an air retention area 142 located at the exhaust hole 141, and a water-blocking and breathable membrane located between the exhaust hole 141 and the air retention area 142. The exhaust hole 141 exhausts excess air during medicine injection to balance internal pressure, and the water-blocking and breathable membrane prevents medicine volatilization or leakage, and also prevents external contaminants from entering and contaminating the medicine solution.

A top of the medicine module 1 is also provided with a sealing plug, and the outer side of the control module 2 is also provided with a transparent window. The transparent window is used to observe the use and remaining status of the internal medicine solution.

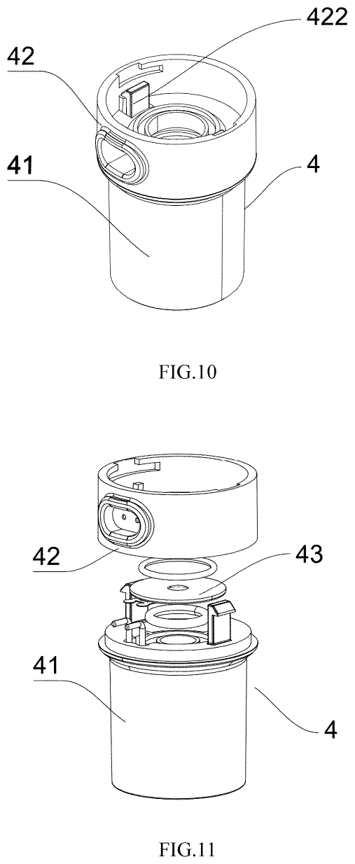

Referring to FIGS. 8 to 11, a core-replaceable atomizer according to a second preferred embodiment of the present invention comprises a liquid medicine storage assembly 3 and an atomization generating assembly 4.

The liquid medicine storage assembly 3 has a built-in filter element 31 for guiding and filtering the liquid medicine, and one end of the atomization generating assembly 4 is connected to the liquid medicine storage assembly 3, and the other end is connected to the patient's inhalation end.

The atomization generating assembly 4 comprises a base 41 for supporting the atomizing element 43 and a connecting tube 42 connected to the patient's inhalation end. One end of the base 41 is connected to the liquid medicine storage assembly 3, and the other end is provided with an atomization area for placing the atomizing element 43. One end of the connecting tube 42 is fixed by connecting to the base 41 to limit the atomizing element 43, and the other end is an interface connected to the patient's inhalation end.

After the device is connected to the breathing circuit, the atomizing element 43 in the atomization generating assembly 4 atomizes the liquid medicine in the liquid medicine storage assembly 3 and then inputs it into the breathing circuit.

Before use, it is necessary to add liquid medicine into the liquid medicine storage assembly 3, then connect it to the breathing circuit, and finally connect the atomizing element 43 to the power supply before it can be turned on for use.

When in use, one end of the filter element 31 for adsorbing the medicine liquid will be in contact with the atomization generating assembly 4, and the medicine liquid will be continuously atomized by the atomization generating assembly 4.

In order to ensure the fit between the filter element 31 and the atomization generating assembly 4 and prevent the filter element 31 from shaking in the liquid medicine storage assembly 3, the size of the filter element 31 is normally larger than the size of the inner cavity of the liquid medicine storage assembly 3, that is, the filter element 31 and the inner cavity fixing structure of the liquid medicine storage assembly 3 are interference fit.

When the medicine needs to be replaced, the liquid medicine storage assembly 3 can be directly replaced, and the atomization generating assembly 4 can be left in the connection of the breathing circuit.

In order to facilitate the filling of medicine, the liquid medicine storage assembly 3 comprises a shell 32 connected and fixed to the base 41. The rear side of the shell 32 is provided with a medicine injection hole, and a sealing cover is provided at the medicine injection hole.

Similarly, as shown in FIGS. 8 to 11, the core-replaceable atomizer of the present solution can also be designed to comprise the detachably connected liquid medicine storage assembly 3 and the atomization generating assembly 4, and the atomized medicine can be replaced by simply replacing the liquid medicine storage assembly 3, eliminating the cleaning and disinfection process of the liquid medicine storage assembly 3, and is easy to operate. The medicine can also be prepared in advance, thereby improving the work efficiency of medical staff and reducing cross infection in the hospital. The relevant structure is as follows.

The liquid medicine storage assembly 3 and the atomization generating assembly 4 are detachably connected, and a plurality of groups of detachable fixing structures are arranged at the connection between the shell 32 of the liquid medicine storage assembly 3 and the base 41 of the atomization generating assembly 4. Each group of the fixing structures includes an “L”-shaped adapter block located on the shell 32 and an embedding area located on the base 41. The positions of the adapter block and the embedding area in each group of fixing structures correspond to each other. When installing or disassembling and replacing the liquid medicine storage assembly 3, the adapter block is attached to the top of the base 41, and is turned into or out of the embedding area by corresponding vertical pressure rotation to complete the installation or disassembly of the liquid medicine storage assembly 3.

In order to better fix the atomizing element 43 in the atomization generating assembly, a plurality of embedding legs 422 are provided at equal angles on one side of the connecting tube 42 close to the base 41, and are fixed after passing through the base 41.

The atomizing element 43 is connected to an external power source through a connection interface on the side wall of the base 41.

In the compensation of the above two solutions, the atomizing element 43 adopts an atomizing sheet with pores, and sealing rings are arranged on the upper and lower sides of the atomizing element 43.

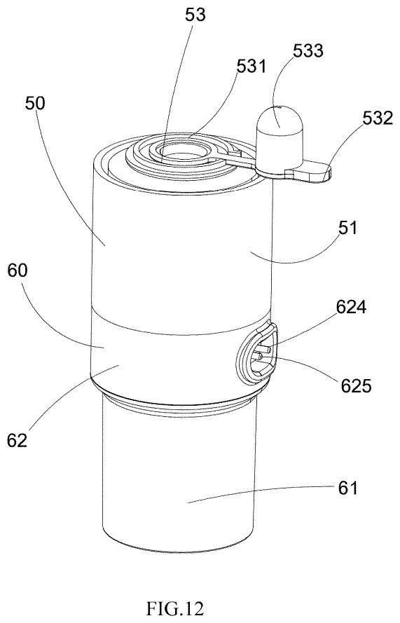

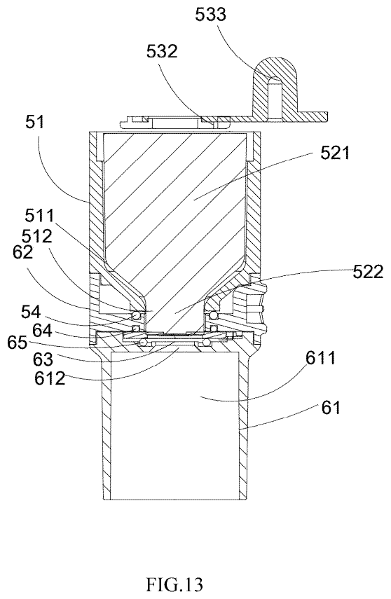

Referring to FIG. 12 to FIG. 14 of the drawings, an atomizer according to a third preferred embodiment of the present invention is illustrated, the atomizer comprises a medicine storage assembly 50 and an atomization assembly 60 for atomizing the medicine liquid delivered from the medicine storage assembly 50.

The medicine storage assembly 50 comprises an outer housing 51 having a storage cavity 511, a medicine liquid transporting element 52 disposed in the outer housing 51, and a medicine injection structure 53 mounted to the outer housing 51 for allowing a syringe needle to inject medicine int the storage cavity 511.

The outer housing 51 serves as the main structural component, enclosing the internal parts of the medicine storage assembly 50. The storage cavity 511 within the outer housing 51 is a designated space for storing the medicine liquid before it is atomized. This centralized storage design reduces the complexity of handling and transferring medicine, potentially improving the safety and accuracy of the dosage.

The medicine liquid transporting element 52, which can be specified to be cotton, plays a significant role in the delivery system by acting as an efficient medium for transporting the medicine liquid from the storage cavity 511 to the atomization assembly 60. Cotton is a porous and absorbent material, ideal for drawing and holding liquid, and its use as a transporting element offers several key advantages:

Cotton fibers have natural capillary properties that allow them to wick liquid upward from the storage cavity 511. This capillary action ensures a consistent flow of medicine to the atomization assembly 60, supporting continuous atomization without interruption. The efficient liquid transfer helps maintain a steady mist output, which is essential for treatments that require consistent dosing over time, like respiratory therapy.

The high absorbency of cotton allows it to hold a significant volume of liquid in relation to its size. This property ensures that medicine can be retained within the cotton element, reducing the risk of leakage or spillage even when the atomizer is moved or tilted.

The absorbent nature of cotton acts as a temporary reservoir, minimizing waste by ensuring that only the necessary amount of liquid is transferred to the atomizer. This enhances dosing precision and reduces medicine waste.

The fiber density and absorption also help to control the liquid flow, reducing the chance of backflow from the atomization assembly 60 back into the storage cavity 511. This containment capability helps in preventing contamination of the remaining medicine in the storage area.

Backflow prevention is particularly beneficial for maintaining the purity of the remaining liquid, which is essential in medical devices where contamination could lead to reduced efficacy or increased health risks.

Cotton is soft and adaptable, allowing it to fit snugly within the storage cavity and maintain contact with the medicine liquid. This consistent fit prevents air bubbles or gaps that could disrupt the liquid flow. The flexibility of cotton ensures a reliable connection between the storage cavity 511 and the atomization assembly 60, making the medicine delivery process continual, smoother and more predictable.

In addition, fibrous structure of cotton can trap small particles, debris, or impurities in the medicine liquid, acting as a preliminary filter before the liquid reaches the atomization assembly. This filtration helps ensure that only purified medicine is atomized.

By filtering out impurities, the atomizer delivers cleaner, safer medicine mist to the patient, which is particularly important in medical treatments where respiratory systems are sensitive to contaminants.

The cotton filter can prevent particulate matter from entering the atomization assembly 60, which can be susceptible to clogging or reduced functionality when exposed to foreign particles. Keeping these contaminants out helps maintain the efficiency of the atomization process and prevents wear or damage to the assembly. This protection ensures a longer lifespan for the atomization assembly 60, reducing maintenance requirements and providing consistent performance over time.

In medical applications, any contaminants in the medicine could potentially cause adverse reactions, especially when the atomized medicine is inhaled. The cotton filter adds a layer of safety by removing potential contaminants that might cause irritation, infection, or other negative effects. The filtration function promotes patient safety by ensuring a cleaner, safer medicine mist, which is critical for patients with respiratory issues or compromised immune systems.

The cotton also brings a significant functional advantage to the atomizer by enabling consistent delivery of medicine liquid independent of the device's orientation. Unlike designs that rely on gravity to transport the liquid, the cotton material's absorbent properties ensure continuous medicine delivery, regardless of whether the atomizer is standing, tilted, or even upside down.

By eliminating the need for the atomizer to remain upright, the cotton transporting element allows users greater flexibility in how they hold and position the device. Patients can use the atomizer in a range of positions that are most comfortable, convenient, or suited to their condition, without worrying about medicine flow interruption.

The medicine injection structure 53 allows for easy injection of medicine into the storage cavity using a syringe. By having a dedicated injection point, users can quickly refill the atomizer without needing to open the main assembly, which can reduce exposure to contaminants and simplify the refilling process.

In this embodiment, the medicine injection structure 53 comprises an injection lid 531 mounted to a top of the outer housing 51 to provide a dedicated port for refilling, a connecting element 532 connected to the injection lid 531 and a sealing pin 533 vertically extended from the connecting element 532 for being inserted into the injection lid 531 for sealing the outer housing 51.

The medicine liquid transporting element 52 of this embodiment comprises a body portion 521 and an end portion 522 extended from the body portion 521, in this embodiment, the end portion 522 is extended through an opening 512 communicated to the storage cavity 511 of the outer housing 51 for feeding the medicine liquid to the atomization assembly 60. A diameter of the end portion 522 is smaller than or equal to a diameter of the body portion 521, in this embodiment, the diameter of the end portion 522 is smaller than the diameter of the body portion 521.

The medicine storage assembly 50 further comprises a sealing member 54 which is a sealing ring provided at the opening 512 and wound around the end portion 522 of the medicine liquid transporting element 52 to provide a sealing effect.

The atomization assembly 60 comprises a base 61, a connecting tube 62 and an atomizing element 63. The atomizing element 63 is mounted on the base 61, and the connecting tube 62 is detachably coupled to the base 61 for supporting the outer housing 51 of the medicine storage assembly 50. The connecting tube 62 can be connected to an air feeding tube, the base 61 can be connected to a a trachea cannula or a breathing mask for feeding the medicament mist to the user. In this embodiment, the connecting tube 62 is coupled between the outer housing 51 and the base 61.

In this embodiment, the end portion 522 of the medicine liquid transporting element 52 is extended into the connecting tube 62 for delivering medicine liquid to the atomizing element 63.

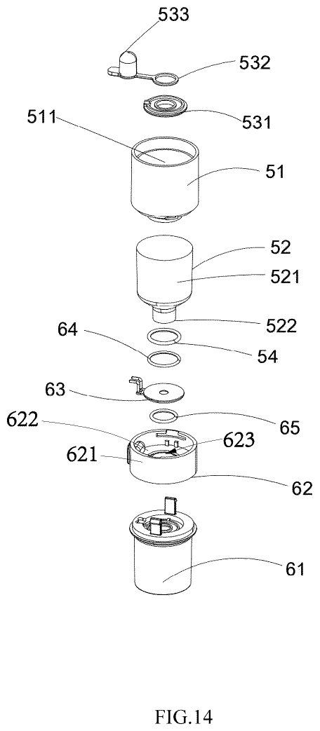

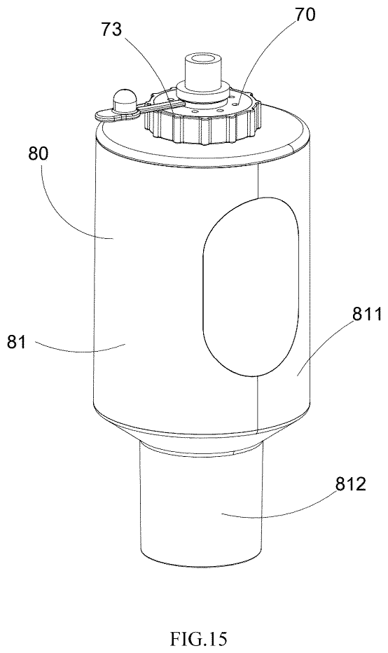

As shown in FIG. 14 of the drawings, when the medicine storage assembly 50 is mounted on the atomization assembly 60, the end portion 522 of the medicine liquid transporting element 52 is aligned with the atomizing element 63 and the distance between the end portion 522 of the medicine liquid transporting element 52 and the atomizing element 63 is preferred to be less than 0.3 mm. This close proximity minimizes the potential for medicine loss due to evaporation or dispersion and ensures that the maximum amount of medicine liquid reaches the atomizing element 63 for conversion into a mist. The precise distance also helps control the flow of liquid, promoting consistent droplet size in the mist, which can improve the delivery of the medicine to the respiratory system of the user.

The connecting tube 62 comprises a tube wall 621, a ring wall 622 extended from an inner side of the tube wall 621 and defines a penetrating hole 623 for allowing the end portion 522 of the medicine liquid transporting element 52 to pass therethrough. The sealing element 54 is provided at the top of the ring wall 622.

The connecting tube 62 comprises a first connecting end portion 624 for connecting to an air feeding tube and a second connecting end portion 625 for connecting to an air recycling tube. Accordingly, air can be fed into the connecting tube 62 and pass through the end portion 522 of medicine liquid transporting element 52, and then reach the atomizing element 63, so as to mix with the mist generated by the atomizing element 63 and finally enter a cavity 611 through a mist outlet 612 of the base 61.

The atomization assembly 60 further comprises a first sealing ring 64 which is mounted at a bottom of the ring wall 622 and is positioned above the atomizing element 63 around the end portion 522 of the medicine liquid transporting element 52, and a second sealing ring 65 which is mounted at a top of the base 61 under the atomizing element 63.

In this embodiment, any suitable detachable connecting manner can be employed to couple the connecting tube 62 with the base 61, such as a snap-fit mechanism, a thread engaging connection, and a magnetic coupling connection. Any suitable detachable connecting manner also can be employed to couple the connecting tube 62 with the outer housing 51 of the medicine storage assembly 50, such as a snap-fit mechanism, a thread engaging connection, and a magnetic coupling connection.

The nebulized medicine can be replaced simply by replacing the medicine storage assembly 50, eliminating the cleaning and disinfection process of the previous plan. The operation is simple, and the medicine can be prepared in advance, which improves the work efficiency of medical staff and reduces cross-infection in the hospital.

The atomizing element 63 is a nebulizer atomizing sheet which is a vibrating mesh with pores. Through the high-frequency resonance of the nebulizer, naturally floating medicine mist is generated.

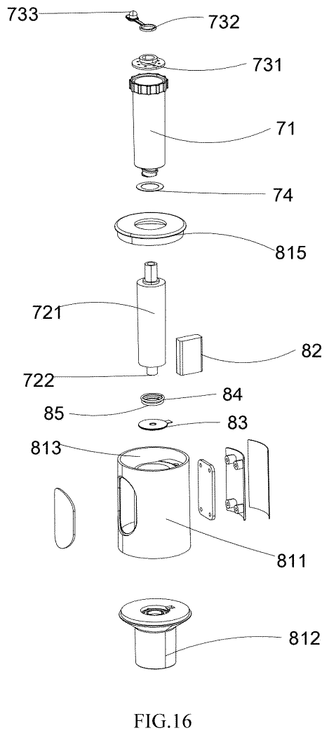

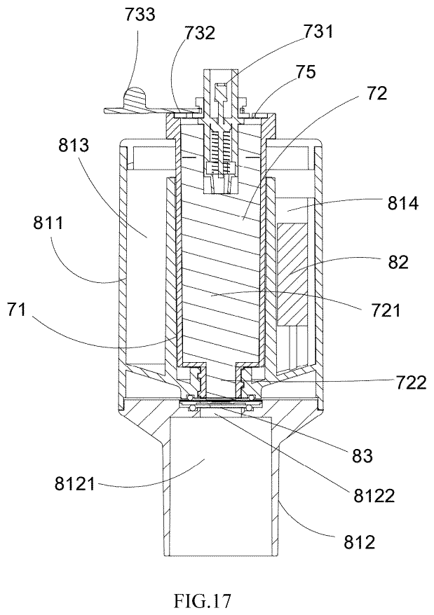

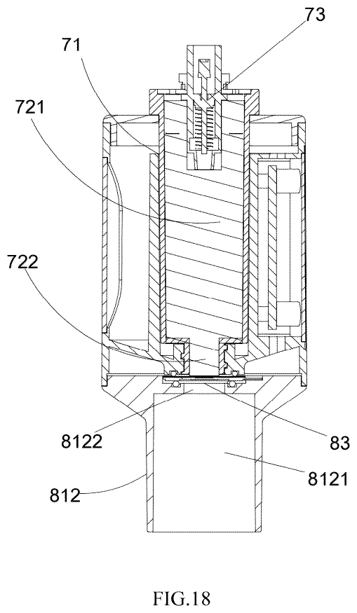

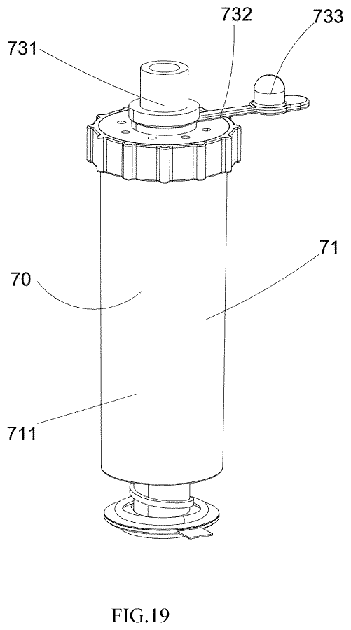

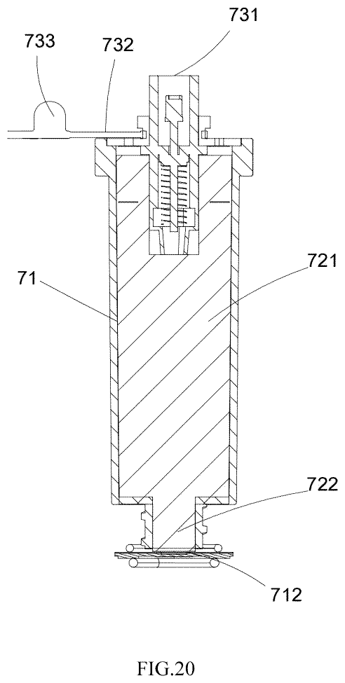

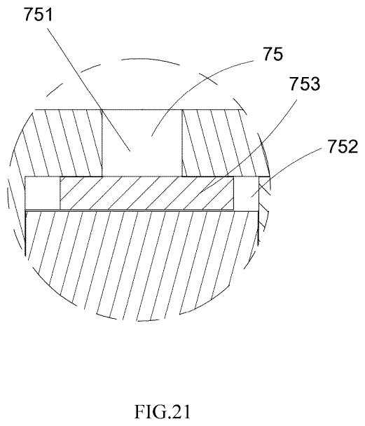

Referring to FIGS. 15 to 21 of the drawings, an atomizer according to a fourth preferred embodiment of the present invention is illustrated. The atomizer comprises a medicine storage assembly 70 and an atomization assembly 80. In this embodiment, the medicine storage assembly 70 is detachably disposed in the atomization assembly 80.

More specifically, the medicine storage assembly 70 comprises a storage housing 71 having a storage cavity 711, a medicine liquid transporting element 72 disposed in the storage cavity 711 and a medicine injection structure 73.

Similarly, the medicine injection structure 73 comprises an injection lid 731 mounted to a top of the storage housing 71 to provide a dedicated port for refilling, a connecting element 732 connected to the injection lid 731 and a sealing pin 732 vertically extended from the connecting element 732 for being inserted into the injection lid 731 for sealing the storage housing 71.

The medicine liquid transporting element 72 can be embodied as a cotton comprising a body portion 721 and an end portion 722 extended from the body portion 721, in this embodiment, the end portion 722 is extended through an opening 712 communicated to the storage cavity 711 of the storage housing 71 for feeding the medicine liquid to the atomization assembly 80. A diameter of the end portion 722 is smaller than or equal to a diameter of the body portion 721.

The medicine storage assembly 70 further comprises a sealing member 74 which is a sealing ring provided at the opening 712 and wound around the end portion 722 of the medicine liquid transporting element 72 to provide a sealing effect.

The atomization assembly 80 comprises a fixing housing 81, a control unit 82 and an atomizing element 83 electrically connected to the control unit 82. The fixing housing 81 comprises a housing body 811 and a base 812 connected to the housing body 811 for mounting the atomizing element 83.

In this embodiment, the storage housing 71 is disposed into an inner cavity 813 of the housing body 811 of the fixing housing 81 so that the storage housing 71 is provided in the fixing housing 81. The fixing housing 81 has a side chamber 814 between the storage housing 71 and the housing body 811 for receiving the control unit 82.

The atomizing element 83, which is preferred to be an atomizing sheet with pores, is mounted at a top of the base 812 and is aligned with the end portion 722 of the medicine liquid transporting element 72, a distance between the atomizing element 83 and the end portion 822 of the medicine liquid transporting element 72 can be less than 0.3 mm.

The atomization assembly 80 may further comprise a first sealing ring 84 which is mounted at a bottom of the housing body 811 and is positioned above the atomizing element 83 around the end portion 722 of the medicine liquid transporting element 72, and a second sealing ring 85 which is mounted at the top of the base 812 under the atomizing element 83.

The base 812 has a cavity 8121 and a mist outlet 8122 under the atomizing element 83, the atomizing element 83 is vibrating to generate medicament mist which is discharged into the cavity 8121 through the mist outlet 8122. The base 812 can be connected to a a trachea cannula or a breathing mask for feeding the medicament mist to the user.

In this embodiment, the medicine storage assembly 70 can be replaced, so that the medicine that needs to be atomized can be replaced conveniently and quickly, and the control module can be retained and reused, saving patients' expenses.

Any suitable detachable connecting manner can be employed to couple the housing body 811 with the storage housing 71, such as a snap-fit mechanism, a thread engaging connection, and a magnetic coupling connection.

The storage housing 71 is further provided with an air exhaust structure 75 having an exhaust hole 751 located at the top wall of the storage housing 71, an air retention area 752 located at the exhaust hole 751, and comprises a water-blocking and breathable membrane 753 located between the exhaust hole 751 and the air retention area 752. The exhaust hole 751 exhausts excess air during medicine injection to balance internal pressure, and the water-blocking and breathable membrane 753 prevents medicine volatilization or leakage, and also prevents external contaminants from entering and contaminating the medicine solution.

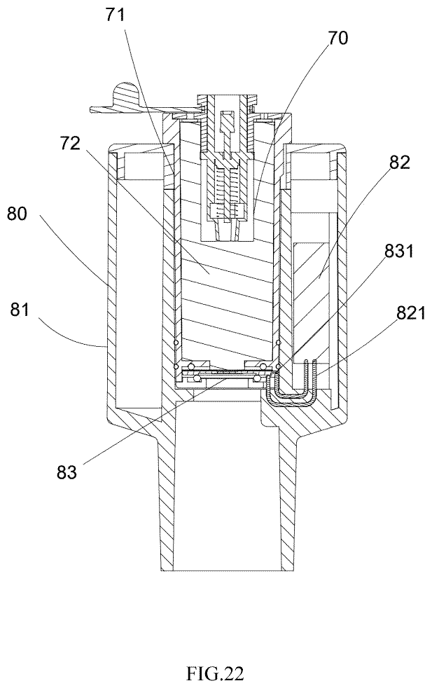

Referring to FIG. 22 of the drawings, as an alternative mode of the above fourth preferred embodiment, the atomizing element 83 comprises two first connecting terminals 831 while the control unit 82 comprises two second connecting terminals 821, the two first connecting terminals 831 can be respectively detachably and electrically connected to the two second connecting terminals 821 when the medicine storage assembly 70 is assembled into the fixing housing 81 of the atomization assembly 80.

In this embodiment, when the atomizing element 83 is detached from the control unit 82, the control unit 82 still can be reused to be electrically connected to a new atomizing element 83.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and are subject to change without departure from such principles. Therefore, this invention includes all modifications encompassed within the spirit and scope of the following claims.

What is claimed is:

1. An atomizer, comprising:
a medicine storage assembly; and
an atomization assembly, wherein said medicine storage assembly is detachably coupled to said atomization assembly.

2. The atomizer according to claim 1, wherein said medicine storage assembly comprises an outer housing having a storage cavity, and a medicine liquid transporting element disposed in said storage cavity of said outer housing, wherein said atomization assembly comprises an atomizing element to align with said medicine liquid transporting element in a manner that said medicine liquid transporting element is arranged to deliver medicine liquid to said atomizing element.

3. The atomizer according to claim 2, wherein said atomization assembly further comprises a base and a connecting tube detchably coupled to said base, wherein said atomizing element is mounted on said base.

4. The atomizer according to claim 3, wherein said connecting tube is detachably coupled with said outer housing for supporting said outer housing.

5. The atomizer according to claim 4, wherein said connecting tube is coupled at a position between said outer housing and said base.

6. The atomizer according to claim 2, wherein said medicine liquid transporting element is a cotton.

7. The atomizer according to claim 5, wherein said medicine liquid transporting element is a cotton which comprises a body portion and an end portion extended from the body portion, wherein said outer housing has an opening, said end portion of said medicine liquid transporting element is extended through said opening which is communicated to said storage cavity of said outer housing for feeding the medicine liquid to said atomizing element.

8. The atomizer according to claim 7, wherein a diameter of said end portion of said medicine liquid transporting element is smaller than or equal to a diameter of said body portion of said medicine liquid transporting element.

9. The atomizer according to claim 8, wherein a distance between said end portion of said medicine liquid transporting element and said atomizing element is less than 0.3 mm.

10. The atomizer according to claim 9, wherein said medicine storage assembly further comprises a medicine injection structure which comprises an injection lid mounted to a top of said outer housing for refilling, a connecting element connected to said injection lid and a sealing pin extended from said connecting element for being inserted into said injection lid.

11. The atomizer according to claim 1, wherein said medicine storage assembly comprises a storage housing having a storage cavity, and a medicine liquid transporting element disposed in said storage cavity of said storage housing, wherein said atomization assembly comprises a fixing housing and an atomizing element disposed in said fixing housing to align with said medicine liquid transporting element in a manner that said medicine liquid transporting element is arranged to deliver medicine liquid to said atomizing element, wherein said storage housing is detachably coupled within said fixing housing.

12. The atomizer according to claim 11, wherein said fixing housing comprises a base and a housing body coupled to said base, wherein said atomizing element is mounted on said base.

13. The atomizer according to claim 12, wherein said storage housing is detachably disposed in said housing body.

14. The atomizer according to claim 13, wherein a side chamber is formed between said storage housing and said housing body, wherein said atomization assembly comprises a control unit which is disposed in said side chamber.

15. The atomizer according to claim 14, wherein said atomizing element is electrically and detachably connected to said control unit.

16. The atomizer according to claim 11, wherein said medicine liquid transporting element is a cotton.

17. The atomizer according to claim 14, wherein said medicine liquid transporting element is a cotton which comprises a body portion and an end portion extended from the body portion, wherein said housing body has an opening, said end portion of said medicine liquid transporting element is extended through said opening which is communicated to said storage cavity of said storage housing for feeding the medicine liquid to said atomizing element.

18. The atomizer according to claim 17, wherein a diameter of said end portion of said medicine liquid transporting element is smaller than or equal to a diameter of said body portion of said medicine liquid transporting element, wherein a distance between said end portion of said medicine liquid transporting element and said atomizing element is less than 0.3 mm.

19. The atomizer according to claim 11, wherein said storage housing is further provided with an air exhaust structure having an exhaust hole located at a top of said storage housing and an air retention area located adjacent to said exhaust hole, wherein said air exhaust structure further comprises a water-blocking and breathable membrane located between said exhaust hole and said air retention area.

20. The atomizer according to claim 19, wherein said medicine storage assembly further comprises a medicine injection structure which comprises an injection lid mounted to a top of said outer housing for refilling, a connecting element connected to said injection lid and a sealing pin extended from said connecting element for being inserted into said injection lid.
